(19) United States
(12) Patent Application Publication
(10) Pub. No.: US 2012/0238463 A1
(43) Pub. Date: Sep. 20, 2012

(54) LINE-1 Hypomethylation as a Biomarker for Early-Onset Colorectal Cancer

(75) Inventors: Ajay Goel, Dallas, TX (US); C. Richard Boland, Dallas, TX (US); Francesc Balaguer, Barcelona (ES)

(73) Assignee: Baylor Research Institute, Dallas, TX (US)

(21) Appl. No.: 13/419,744

(22) Filed: Mar. 14, 2012

Related U.S. Application Data
(60) Provisional application No. 61/454,130, filed on Mar. 18, 2011.

(51) Int. Cl. C12Q 1/68; C40B 30/04; C40B 30/00

(52) U.S. Cl. 506/9; 435/6.11; 506/7

(57) ABSTRACT

A method for detecting an early-onset of colorectal cancer in a human subject is disclosed herein. The method comprises the steps of: (i) identifying the human subject suspected of suffering from a colorectal cancer, (ii) obtaining one or more biological samples from the human subject; (iii) determining a LINE-1 methylation level for the one or more biological samples; and (iv) comparing the LINE-1 methylation level to a LINE-1 methylation control level, wherein a higher degree of the LINE-1 methylation level is indicative of an early-onset colorectal cancer.

### CROSS-REFERENCE TO RELATED APPLICATIONS

This application claims priority to U.S. Provisional Application Ser. No. 61/454,130, filed Mar. 18, 2011, the entire contents of which are incorporated herein by reference.

### STATEMENT OF FEDERALLY FUNDED RESEARCH

This invention was made with U.S. Government support under Contract Nos. R01 CA72851 and CA129286 awarded by the National Cancer Institute (NCI)/National Institutes of Health (NIH). The government has certain rights in this invention.

### TECHNICAL FIELD OF THE INVENTION

The present invention relates in general to the field of cancer prediction, detection, diagnosis, monitoring and treatment, and more particularly, to methods for detecting early-onset colorectal cancers (CRCs) based on hypomethylation of LINE-1.

### REFERENCE TO A SEQUENCE LISTING

The present application includes a Sequence Listing which has been submitted in ASCII format via EFS-Web and is hereby incorporated by reference in its entirety. Said ASCII copy, created on Mar. 12, 2012, is named BHCS1124_Sequence_Listing_ST25.txt and is 882,499 bytes in size.

### BACKGROUND OF THE INVENTION

Without limiting the scope of the invention, its background is described in connection with markers for detection and diagnosis of early onset cancers, including colorectal cancers.

U.S. Patent Application No. 20110028332 (Kuroda et al. 2011) provides a marker, a test method, and a test kit which can detect the onset of breast cancer that cannot be detected by palpation or mammography examination or breast cancer in an early stage (clinical stage 0), which are simple, and which have high reliability. The marker in the Kuroda invention is a micro-RNA that is found in serum or plasma. More specifically, the marker contains at least a micro-RNA that is present in the serum or the plasma at a significantly reduced level after the onset of breast cancer, or during or after an early stage (during or after clinical stage 0) of breast cancer compared with that before the onset of breast cancer or before the early stage (before clinical stage 0) of breast cancer.

U.S. Pat. No. 7,547,771 issued to Blumenfeld et al. (2011) discloses the genomic sequence and cDNA sequences of the PCTA-1 gene. The Blumenfeld invention also concerns biallelic markers of the PCTA-1 gene and the association established between these markers and prostate cancer. The invention provides means to determine the predisposition of individuals to prostate cancer as well as means for the diagnosis of prostate cancer and for the prognosis/detection of an eventual treatment response to agents acting against prostate cancer.

U.S. Patent Application No. 20090068660 (Hoon and Sunami, 2009) relates to a method of detecting LINE-1 (long interspersed nucleotide elements-1) DNA either methylated or unmethylated at the promoter region in a tissue or body fluid sample from a subject. Also disclosed are methods of using LINE-1 DNA as a biomarker for diagnosing, predicting, and monitoring cancer progression and treatment.

### SUMMARY OF THE INVENTION

The present invention provides a method for predicting, detecting, diagnosing or monitoring an early-onset of colorectal cancer in a human subject by identifying the human subject suspected of suffering from a colorectal cancer; obtaining one or more biological samples from the human subject; determining a LINE-1 methylation level for the one or more biological samples; and comparing the LINE-1 methylation level to a LINE-1 methylation control level, wherein a lower degree of the LINE-1 methylation level is indicative of an early-onset colorectal cancer.

The present invention also provides a biomarker for predicting, detecting, diagnosing or monitoring an early-onset of colorectal cancer in a human subject having a biomarker to determine a methylation level of LINE-1, wherein a lower methylation level of LINE-1 is indicative of an early-onset colorectal cancer in the human subject. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In another aspect, the LINE-1 methylation level is determined by, for example, amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing. In another aspect, the LINE-1 methylation level is determined by quantitative bisulfite pyrosequencing. In another aspect, the LINE-1 methylation level is determined by quantitative bisulfite pyrosequencing using the nucleic acids of SEQ ID NOS: 1 to 20.

The present invention provides a kit for predicting, detecting, diagnosing or monitoring an early-onset of colorectal cancer in a human subject having a biomarker detecting reagent for measuring a LINE-1 methylation level in a sample; and instructions for the use of the biomarker detecting reagent in diagnosing the presence of early-onset of colorectal cancer, wherein the instructions comprise providing step-by-step directions to compare the LINE-1 methylation level in the sample with a LINE-1 methylation control level. In one aspect the sample is selected from the group consisting of a tissue sample, a fecal sample, a cell homogenate, a blood sample, one or more biological fluids, or any combinations thereof. In another aspect the LINE-1 methylation control level is obtained from the sample from a healthy subject, wherein the healthy subject is a human subject not suffering from early-onset colorectal cancer. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In another aspect, the LINE-1 methylation level is determined by quantitative bisulfite pyrosequencing. In another aspect, the LINE-1 methylation level is determined by quantitative bisulfite pyrosequencing using the nucleic acids of SEQ ID NOS: 1 to 20.

The present invention provides a method for selecting a cancer therapy for a patient diagnosed with early-onset of colorectal cancer by determining a methylation level of LINE-1 in a biological samples of the subject, wherein the methylation level of LINE-1 is indicative of early-onset of colorectal cancer; and selecting the cancer therapy based on the determination of the presence of early-onset of colorectal cancer in the subject. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In another aspect, the LINE-1 methylation level is determined by, for example, amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing. In another aspect, the LINE-1 methylation level is determined by quantitative bisulfite pyrosequencing. In another aspect, the LINE-1 methylation level is determined by quantitative bisulfite pyrosequencing using the nucleic acids of SEQ ID NOS: 1 to 20.

The present invention also provides a method of performing a clinical trial to evaluate a candidate drug believed to be useful in treating early-onset of colorectal cancer by a) determining the presence of an early-onset of colorectal cancer by a method comprising the steps of: determining an overall LINE-1 methylation level in one or more cells obtained from a biological sample of the subject, wherein a lower overall LINE-1 methylation level compared to a reference control is indicative of an early-onset of colorectal cancer; b) administering a candidate drug to a first subset of the patients, and a placebo to a second subset of the patients; c) repeating step a) after the administration of the candidate drug or the placebo; and d) monitoring a change in the overall LINE-1 methylation level as compared to any reduction occurring in the second subset of patients, wherein a statistically significant reduction indicates that the candidate drug is useful in treating said disease state. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In one aspect, the biological samples are selected from the group consisting of a tissue sample, a fecal sample, a cell homogenate, a blood sample, one or more biological fluids, or any combinations thereof. In another aspect, the LINE-1 methylation level is higher than an Alu methylation level. In another aspect, the LINE-1 methylation level is determined by, e.g., amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing. In another aspect, the LINE-1 methylation level is determined by quantitative bisulfite pyrosequencing. In another aspect, the LINE-1 methylation level is determined by quantitative bisulfite pyrosequencing using the nucleic acids of SEQ ID NOS: 1 to 20.

Yet another embodiment of the invention is a method of using a pharmacodynamic (PD) biomarker for determining a pharmacological response to a treatment of early-onset of colorectal cancer, the method comprising: determining an overall LINE-1 methylation level in one or more cells obtained from a first biological sample of a subject, wherein a lower overall LINE-1 methylation level compared to a normal sample from the subject that is not suspected of having cancer, is indicative of an early-onset of colorectal cancer; administering a drug to the subject at a first time, repeating the step of determining an overall LINE-1 methylation level in one or more cells obtained from a second biological sample from the subject at a second time; and comparing the overall LINE-1 methylation at the first and the second time, wherein a statistically significant reduction in LINE-1 methylation indicates that the drug is useful in treating said disease state.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the average methylation in the CRCs was 59.97% (standard deviation, 6.57), which followed a normal distribution;

FIG. 2 shows LINE-1 methylation analysis by bisulfite pyrosequencing in different CRC subsets. Bisulfite pyrosequencing of LINE-1 in colorectal tissues; Normal mucosa (n=32), early-onset CRC from Argentina (n=116), early-onset CRC from Spain (n=70), older onset CRC with microsatellite stability (MSS; n=89), older onset CRC with microsatellite instability (MSI) associated with MLH1 promoter hypermethylation (n=46) and Lynch syndrome CRCs (n=20). The black horizontal bar indicates the mean methylation level.

FIG. 3 shows Kaplan-Meier survival curves depicting the effect of LINE-1 (left panel) and mismatch repair deficiency (right panel) on 3-year overall survival in early-onset CRC patients. Vertical tick marks indicate censored events. On the left graph, the green line represents survival in CRCs with LINE-1 hypomethylation (<65%) and the blue line represents LINE-1 methylation >65%. In the right graph, the green line represents survival in patients whose tumors had DNA MMR deficiency, and the blue line represents that in patients with DNA MMR-proficient tumors.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Abbreviations: CRC, colorectal cancer; MSI, microsatellite instability; MSS, microsatellite stability; LINE-1, long interspersed nucleotide element-1.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions which are characterized by cancer of cells of the duodenum and small intestine (jejunum and ileum).

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., a-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH₂Cl₂, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me2S (source: infection; diagnosis: trench mouth).

Major classes of cancer biomarkers based on clinical utility and application include the following: (1) “diagnostic biomarkers” that are used to: (i) determine if the patient has cancer, and (2) define the type of cancer of the patient. Diagnostic biomarkers can also be used to detect and define recurrent disease after primary therapy. (2) “Prognostic biomarkers” are used to indicate a likely course of the disease. Prognostic biomarkers can reflect, for example, the metastatic state or potential and/or the likely growth rate of the tumor, and are used to estimate patient outcome without consideration of the treatment given. (3) “Predictive biomarkers” are used to identify subpopulations of patients who are most likely to respond to a given therapy. (4) “Pharmacodynamic” or “pharmacological” biomarkers (sometimes referred to as PD biomarkers) can help identify which drug dose to use for an individual. Finally, biomarkers can also be used to monitor a patient's response to treatment. Once a patient begins treatment with a drug, the biomarkers of the present invention can be used to monitor the patient's response, and if necessary, the treatment regiment (drug or dose) can be modified. The biomarkers of the present invention can be used in any of these forms.

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “kit” or “testing kit” denotes combinations of reagents and adjuvants required for an analysis. Although a test kit consists in most cases of several units, one-piece analysis elements are also available, which must likewise be regarded as testing kits.

Methylation analysis can be conducted by any of a number of currently known (or future) methods, that are generally divided into those performed by, e.g., enzymatic digestion, chemical reactions or affinity enrichment. These can be further divided into those that are specific for a methylated sequence or loci, gel based analysis, array based analysis, or a variety of old and new sequencing methodologies. Examples of methods for methylation determination include, but are not limited to: amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing.

Colorectal cancer (CRC) is an important public health problem and represents the second most frequent cancer and the second greatest cause cancer-related mortality in most of the developed world. Each year, one million people develop CRC, and 40-50% of them will die within 5 years of diagnosis. CRCs are highly heterogeneous both histopathologically, and at the molecular and genetic level. It appears that the biology and response to therapies is equally diverse. Understanding the molecular mechanisms of colorectal carcinogenesis is essential for the development of new strategies for prevention, diagnosis, treatment and prognosis. Although CRC has been a major focus of attention for basic and clinical research during the last 25 years, we still lack robust biomarkers that can be used for diagnosis and treatment of CRC.

The peak incidence of CRC is between 60-70 years old; however up to 10% of all cases occur before age 50. Moreover, recent epidemiological studies suggest that the incidence of early-onset CRC is increasing, representing an important clinical challenge². Early-onset CRC often presents with advanced stage tumors, which contributes to a higher rate of mortality³. Since young people are not included in CRC screening programs, there is an urgent need to understand the biology of early-onset tumors, which could facilitate earlier detection and treatment of these cancers.

Although early-onset CRC raises the possibility of a hereditary risk factor, the known non-polyposis hereditary CRC syndromes (Lynch Syndrome and MUTYH-associated CRC) represent no more than 15-20% of cases in this group⁴⁻⁶. Lynch Syndrome accounts for about 3% of all CRC cases, and is caused by germline mutations of the DNA mismatch repair (MMR) genes (MLH1, MSH2, MSH6 and PMS2)⁷. Lynch Syndrome is characterized by early-onset cancers arising in the colorectum and other organs, and there are currently several strategies and algorithms to predict the presence of a germline mutation in one of the MMR genes⁸⁻¹¹. Biallelic mutations in the MUTYH gene (a member of the base excision repair system) accounts for <1% of all CRC, and usually causes an attenuated form of polyposis, although 30% of these patients can manifest as a non-polyposis CRC¹². Identifying individuals with germline mutations that predispose to CRC has significant implications for the clinical management of affected individuals and for their relatives.

The remaining 75-80% of early-onset CRC represents another group in which the genetic etiology has not yet been discovered. In contrast to CRC on older individuals, early-onset CRC is often characterized by more advanced stage, distal location (especially in rectum), mucinous and poorly differentiated tumors with signet ring cells, and a poorer prognosis^{4, 13, 14}. The majority of these cancers do not show microsatellite instability (MSI), but rather are microsatellite stable (MSS). The molecular basis for the biological and behavioral differences in early-onset CRC is unclear.

Recent epidemiological studies have shown that the incidence of early-onset colorectal cancer (CRC) is increasing, representing an important clinical challenge. Early-onset CRC often presents with advanced stage tumors, which contributes to a higher rate of mortality. Since young people are not usually included in CRC screening programs, there is an urgent need to understand the biology of early-onset tumors, which might facilitate earlier detection and treatment of these cancers.

Methylation of LINE-1 elements constitutes a surrogate marker for global DNA methylation. LINE-1 hypomethylation has been recently recognized as an independent factor for increased cancer-related mortality in CRC patients. A large cohort of early-onset CRCs was studied and it was found that LINE-1 hypomethylation in these tumors constitutes a significant feature compared with older-onset CRC, which suggests a distinct molecular subtype. Thus, LINE-1 methylation status can be used as a predictive and prognostic biomarker for young people with CRC.

The present invention provides a unique indicator of early-onset colorectal cancer (CRC), specifically in the increase in hypomethylation of LINE-1. Early-onset colorectal cancer (e.g., onset before 50 years of age) accounts for up to 10% of all colorectal cancer. In contrast to older cases, early-onset colorectal cancer is characterized by more advanced stage, distal location (especially in rectum) and poor prognosis. The present inventors have shown that the hereditary syndromes, Lynch syndrome and MUTYH-associated colorectal cancer, account for only 15-20% of the cases, and the majority do not show microsatellite instability (MSI) and are hence microsatellite stable (MSS).

Genome-wide DNA hypomethylation has been recognized as a common epigenetic change in colorectal cancers, which associates with the activation of certain proto-oncogenes and may facilitate chromosomal instability. Hypomethylation of LINE-1 repetitive sequences is a surrogate marker for global DNA hypomethylation, and is also an independent factor for increased cancer-related mortality and overall mortality in colorectal cancer patients. However, the methylation status of LINE-1 elements in early-onset colorectal cancer compared to older-onset colorectal cancer remains unknown.

The present inventors analyzed a cohort of non-polyposis colorectal cancer diagnosed at ages before the age of 50 years recruited in Argentina (Dr. C.B. Udaondo Hospital, n=115) and Spain (Hospital Clinic of Barcelona, Hospital of Donostia; n=70). As a control group, a population-based cohort of sporadic colorectal cancer aged older than 50 years was used and recruited in Spain (EPICOLON I study), and categorized the tumors by the presence of sporadic MSI (due to somatic promoter hypermethylation of MLH1, n=46) or sporadic MSS (n=89) cancers. In addition, we analyzed a group of Lynch syndrome colorectal cancers recruited at Baylor University Medical Center at Dallas (n=20). The methylation status of LINE-1 repetitive elements in various groups of tumor specimens was analyzed by quantitative bisulfite pyrosequencing.

The mean LINE-1 methylation levels (±standard deviation, SD) in the four study groups were: early-onset colorectal cancer, 56.57% (±8.6); sporadic MSI, 67.14% (±6.2); sporadic MSS, 65.14% (±6.2) and Lynch syndrome, 66.3% (±4.5). Early-onset colorectal cancer displayed a significantly lower degree of LINE-1 methylation than any other group (sporadic MSI, p<0.0001; MSS, p<0.0001; Lynch syndrome, p<0.0001). This difference remained significant for both cohorts of early-onset colorectal cancer enrolled in Argentina and Spain.

These findings demonstrate that a higher degree of LINE-1 hypomethylation is a unique feature of early-onset colorectal cancers, and distinguishes them from older colorectal cancers. Since LINE-1 hypomethylation is a surrogate marker for increased chromosomal instability, these data provide a novel and previously unrecognized explanation for some of the biological differences underpinning early-onset colorectal cancers. In one embodiment, the present invention provides a method of diagnosing and treating early-onset colorectal cancers by examining LINE-1 hypomethylation.

Early-onset colorectal cancer (CRC) represents a clinically distinct form of CRC that is often associated with a poor prognosis. Methylation levels of genomic repeats such as LINE-1 elements have been recognized as independent factors for increased cancer-related mortality. The methylation status of LINE-1 elements in early-onset CRC has not been analyzed previously. As such, 343 CRC tissues and 32 normal colonic mucosa samples were analyzed, including two independent cohorts of CRC diagnosed ≦50 years old (n=188), a group of sporadic CRC >50 years (MSS n=89; MSI n=46), and a group of Lynch syndrome CRCs (n=20). Tumor mismatch repair protein expression, microsatellite instability status, LINE-1 and MLH1 methylation, somatic BRAF V600E mutation, and germline MUTYH mutations were evaluated. Briefly, Mean LINE-1 methylation levels (±SD) in the five study groups were: early-onset CRC, 56.6% (8.6); sporadic MSI, 67.1% (5.5); sporadic MSS, 65.1% (6.3); Lynch syndrome, 66.3% (4.5) and normal mucosa, 76.5% (1.5). Early-onset CRC had significantly lower LINE-1 methylation than any other group (p<0.0001). Compared to patients with ≦65% LINE-1 methylation in tumors, those with >65% LINE-1 methylation had significantly better overall survival (p=0.026, log rank test). It was found that LINE-1 hypomethylation constitutes an important feature of early-onset CRC, and suggests a distinct molecular subtype. As such, LINE-1 methylation status can be used as a prognostic biomarker for patients, e.g., young patients, with CRC.

Genome-wide DNA hypomethylation is a frequent epigenetic alteration that is an early event in CRC and has been associated with the activation of certain proto-oncogenes (i.e., MET)¹⁵ and the presence of chromosomal instability^{16, 17}. Global DNA hypomethylation can be measured indirectly by assessing the methylation status of long interspersed nucleotide element-1 (LINE-1) repeat sequences¹⁸. The pyrosequencing assay for LINE-1 methylation has been found to be quantitative, robust and reproducible⁹. The degree of LINE-1 hypomethylation has been recognized as an independent factor for increased cancer-related mortality and overall mortality in CRC patients²⁰. Although it has been suggested that LINE-1 hypomethylation is associated with CRC in younger patients²¹, the specific association between methylation status of LINE-1 elements and early-onset CRC has not been analyzed.

This study characterized the clinical, histological, and molecular features of a large cohort of early-onset CRCs in the context of the methylation status of LINE-1 elements. Our results indicate that LINE-1 hypomethylation in these tumors constitutes a unique and specific feature, which is suggestive of a distinct molecular subtype in these colorectal neoplasms. Our findings suggest that LINE-1 methylation status could be used as a prognostic biomarker for young people with CRC.

Patients and Methods. 343 CRCs from different clinicopathological groups, and 32 normal colonic mucosa samples were analyzed. A cohort of 118 retrospectively recruited CRC patients ≦50 years old was included from the Oncology Section of the Argentine Public Hospital of Gastroenterology between 1993 and 2009. Patients with colorectal polyposis or inflammatory bowel disease were excluded. Demographic and clinicopathological features were collected from each patient's medical history, and family history of cancer in first and second degree relatives was obtained by personal interview. The median follow-up time was 39 months (range, 1.5-195 months). For the LINE-1 methylation analyses, as a validation group, which included a previously described cohort of 70 patients with CRC diagnosed ≦50 years old treated at two Spanish centers (Hospital Clinic of Barcelona and Hospital of Donostia) between 1995-2007⁴. Also included was a population-based cohort of sporadic CRCs>50 years recruited in Spain (Epicolon I study)⁹ categorized by the presence of MSI (“sporadic MSI” due to somatic MLH1 promoter hypermethylation [n=46], and “sporadic MSS” [n=89]); and a group of Lynch syndrome CRCs recruited at Baylor University Medical Center at Dallas (n=20). Normal colonic mucosa from 32 individuals undergoing colonic surgery for reasons other than cancer (i.e. diverticulosis) were analyzed histologically. The study was approved by the Ethics Committee of each participating center, and a written informed consent was obtained from all patients.

DNA isolation. Genomic DNA from each patient was extracted from formalin-fixed paraffin-embedded (FFPE) microdissected tumor tissues using the QiaAmp Tissue Kit (Qiagen, Courtaboeuf, France) according to the manufacturers' instructions. Peripheral blood DNA was extracted using the QiaAmpDNA blood Mini Kit (Qiagen, Courtaboeuf, France).

Tumor mismatch repair protein expression. One block of FFPE tumor tissue was selected per case and immunostaining was performed using standard protocols. The following mouse monoclonal antibodies were used: anti-MLH1 (clone G168-728, diluted 1:250, PharMingen, San Diego, Calif.), anti-MSH2 (clone FE11, diluted 1:50, Oncogene ResearchProducts, Cambridge, Mass.), anti-MSH6 (clone GRBP.P1/2.D4, diluted 1:200; Serotec Inc, Raleigh, N.C.) and anti-PMS2 (clone A16-4, diluted 1:200, BD PharMingen, San Diego, Calif.). A tumor was deemed negative for protein expression only if the neoplastic epithelium lacked nuclear staining, while non-neoplastic epithelial or stromal cells retained normal expression of that protein.

Tumor microsatellite instability analysis. MSI analysis was carried out using five mononucleotide repeat microsatellite targets (BAT-25, BAT26, NR-21, NR-24 and NR-27) in a pentaplex PCR system. Primer sequences have been described previously and area incorporated herein by reference²². Tumors with instability at ≧3 these markers were classified as microsatellite unstable (MSI) and those showing instability at ≦2 markers as microsatellite stable (MSS). The researchers scoring immunostaining were blinded to the MSI results, and vice versa.

Methylation analyses. DNA was modified with sodium-bisulfite using the EZ Methylation Gold Kit (Zymo Research, Orange, Calif.). Methylation of LINE-1 sequences and the promoter of MLH1 was analyzed by quantitative bisulfite pyrosequencing as described previously²³. Primers are detailed in Table 5.

Germline MUTYH gene mutation analysis. All patients were screened for the two most prevalent MUTYH mutations in Caucasian populations (p.G393D and p.Y176C) by pyrosequencing. Primers are detailed in Table 5. In heterozygotes for any of these mutations, the coding region and exon-intron boundaries of the MUTYH gene were screened by SSCP with sequencing of abnormal band shifts, as described previously¹².

Somatic BRAF V600E mutation analysis. The BRAF V600E mutational analysis was performed by pyrosequencing. The PCR and sequencing primers are detailed in Table 5.

Statistical analysis. Data were analyzed using SPSS v17 software. Quantitative variables were analyzed using Student's test. Qualitative variables were analyzed using either the Chi Square test or the Fisher's test when appropriate. The Mann Whitney test was used to compare LINE-1 values. Overall survival associated with clinicopathological and molecular variables (tumor stage, MMR deficiency, tumor location, family history of CRC, tumor differentiation, mucin status, tumor infiltrating lymphocytes and LINE-1 methylation) were calculated by using the Kaplan-Meier method (log rank test). A two sided p-value of <0.05 was regarded as significant.

Patient's characteristics. A total of 118 patients were recruited with early-onset CRC. Clinicopathological features are shown in Table 1. The mean age at diagnosis was 37 years (standard deviation (SD), 8.25), and 61 (51.7%) patients were female. In 34 (28.8%) the tumor was proximal to the splenic flexure, 35 (29.6%) were in the distal colon, and 49 (41.6%) were in the rectum. At presentation, 22 (18.6%) patients had 1-10 synchronous adenomas; 18 presented with 1-5 adenomas and 4 patients had 6-10 adenomas. Three cases (2.5%) had a synchronous tumor (2 CRC and 1 neuroendocrine tumor in the appendix), and 5 (4.2%) developed a metachronous tumor during follow-up (4 CRC and 1 urothelial carcinoma). The majority of cases (77; 65.3%) were diagnosed at advanced stages (IIIIV). Poorly differentiated tumors were seen in 15 (13.1%) patients, 41 (34.7%) had mucinous features and 65 (55%) had pathological features suggestive of the MSI phenotype, with one or more of the following: signet ring cells, Crohn's-like lymphocytic reaction, tumor infiltrating lymphocytes, medullary growth pattern, or anaplastic features. More than 85% (n=100) of the patients had experienced abdominal pain prior to diagnosis, 83 (70%) presented with an alteration in bowel habits, 71 (60%) had rectal bleeding and weight loss, 34 (29%) had iron deficiency anemia, 18 (15.5%) presented with bowel obstruction, and 6 (5%) with perforation. The average delay between initial symptoms and CRC diagnosis was 6.5±5 months. Fifteen patients (12.7%) had a family history of CRC or another Lynch syndrome-associated neoplasm in first or second-degree relatives. Three patients met Amsterdam I criteria, 3 patients met Amsterdam II criteria, 4 patients had one first degree relative with CRC, 3 patients had two or more second degree relatives with CRC, and 2 patients had one second degree relative with CRC.

TABLE 1
Clinical, pathological and molecular features of patients with mismatch repair deficiency

| Clinical, pathological or | Cohort | | MMR deficient¹ | | MMR proficient² | | |
|---|---|---|---|---|---|---|---|
| molecular feature | N = 118 | | N = 27 (22.9%) | | N = 91 (77.1%) | | p-value |
| Age at diagnosis, mean (standard deviation) | 37 | (8.25) | 35 | (10.06) | 38 | (7.55) | 0.23 |
| Range | (29-45) | | (25-45) | | (30-45) | | |
| Sex, n (%) | | | | | | | |
| Female | 61 | (51.7) | 13 | (48.1) | 48 | (52.7) | 0.67 |
| Male | 57 | (48.3) | 14 | (51.9) | 43 | (47.3) | |
| Tumor location, n (%) | | | | | | | |
| Proximal to splenic flexure | 34 | (28.8) | 16 | (59.3) | 18 | (19.8) | 0.0001 |
| Distal to splenic flexure | 84 | (71.2) | 11 | (40.7) | 73 | (80.2) | |
| Synchronous or metachronous CRC, n (%) | | | | | | | |
| yes | 6 | (5.1) | 3 | (11.1) | 3 | (3.3) | 0.132 |
| no | 112 | (94.9) | 24 | (88.9) | 96.7) | | |
| Synchronous adenomas, n (%) | | | | | | | |
| 0 | 81 | (68.6) | 18 | (66.7) | 63 | (69.2) | 0.589 |
| 1-5 | 18 | (15.2) | 6 | (22.2) | 12 | (13.2) | |
| 6-10 | 4 | (3.4) | 1 | (3.7) | 3 | (3.3) | |
| Incomplete colonoscopy | 15 | (12.8) | 2 | (7.4) | 13 | (14.3) | |
| Synchronous hyperplastic polyps, n (%) | | | | | | | |
| 0 | 95 | (80.5) | 24 | (86.2) | 71 | (78.0) | 0.644 |
| 1-5 | 7 | (6) | 1 | (6.9) | 6 | (6.6) | |
| 6-10 | 1 | (0.70) | 0 | (0) | 1 | (1.1) | |
| Incomplete colonoscopy | 15 | (12.8) | 2 | (6.9) | 13 | (14.3) | |
| Family history of CRC or other Lynch syndrome | | | | | | | |
| associated neoplasia³, n (%) | | | | | | | |
| Yes | 15 | (12.7) | 5 | (18.5) | 10 | (11.0) | 0.30 |
| No | 103 | (87.3) | 22 | (81.5) | 81 | (89.0) | |
| TNM tumor stage, n (%) | | | | | | | |
| I-II | 41 | (34.7) | 14 | (51.9) | 27 | (29.7) | 0.03 |
| III-IV | 77 | (65.3) | 13 | (48.1) | 64 | (70.3) | |
| Tumor differentiation, n (%) | | | | | | | |
| Well or moderate | 100 | (86.9) | 24 | (88.9) | 76 | (86.3) | 1 |
| Poor | 15 | (13.1) | 3 | (11.1) | 12 | (13.7) | |
| Mucinous component, n (%) | | | | | | | |
| ≧50% | 41 | (34.7) | 17 | (63) | 24 | (26.4) | 0.0001 |
| <50% | 77 | (65.3) | 10 | (37) | 67 | (73.6) | |
| Tumor infiltrating lymphocytes, n (%) | | | | | | | |
| Yes | 26 | (22.8) | 16 | (59.3) | 10 | (11.5) | 0.0001 |
| No | 88 | (77.2) | 11 | (40.7) | 77 | (88.5) | |
| Medullary growth pattern, n (%) | | | | | | | |
| Yes | 11 | (9.4) | 3 | (11.1) | 8 | (8.9) | 0.714 |
| No | 106 | (90.6) | 24 | (88.9) | 82 | (91.1) | |
| Tumors with Crohn's reaction, n (%) | | | | | | | |
| Yes | 12 | (10.6) | 5 | (18.5) | 7 | (8.1) | 0.154 |
| No | 101 | (89.4) | 22 | (81.5) | 79 | (91.9) | |
| Pathology suggestive of MSI⁴, n (%) | | | | | | | |
| Yes | 65 | (55) | 22 | (81.5) | 43 | (47.2) | 0.002 |
| No | 53 | (45) | 5 | (18.5) | 48 | (52.8) | |
| Somatic BRAF mutations, n (%) | | | | | | | |
| Wild-type | 108 | (96.4) | 25 | (96.2) | 83 | (96.5) | 1 |
| Mutated | 4 | (3.6) | 1 | (3.8) | 3 | (3.5) | |
| LINE-1 methylation, mean (standard deviation) | 59.97 | (6.57) | 61.26 | (6.13) | 59.7 | (6.68) | 0.244 |
| Progression/recurrence | | | | | | | |
| Yes | 46 | (39) | 6 | (22.2) | 40 | (44) | 0.042 |
| No | 72 | (61) | 21 | (77.8) | 51 | (56) | |

| | | | | |
|---|---|---|---|---|
| Three-year overall survival | 84.7%% | 96.3% | 83.5% | 0.115 |

¹MSI-H and/or loss of expression of MMR proteins by immunohistochemistry
²MSS and normal expression of MMR proteins by immunohistochemistry
³Including first and second degree relatives; Lynch syndrome-associated neoplasia includes: endometrium, stomach, ovaries, urinary tract, small intestine, pancreas, bile ducts, brain or sebaceous glands.
⁴Signet ring cells and/or Crohn's-like lymphocytic reaction and/or tumor infiltrating lymphocytes and/or medullary growth pattern and/or anaplastic tumor

Follow-up was available on all 118 patients, ranging from 1.5 to 195 months, with a mean of 39 months. The 3-year survival rate for all 118 patient in this series was 84.7%; 46 patients (39%) relapsed or had progression of disease, 22 (18.6%) died, and 3 patients were lost to follow-up. Advanced tumor stage was significantly associated with a worse 3-year overall survival (stages I-II: 92.9% vs. stages 82.9%; p=0.046, log rank test) and a trend was observed for better survival in patients with mucinous tumors (95.1% vs. 82.9%; p=0.077, log rank test) or with tumor infiltrating lymphocytes (96.2% vs. 85.2%; p=0.16, respectively; log rank test).

Mismatch repair deficiency analysis. MMR deficiency was evaluated by MSI analysis and immunohistochemistry, and was defined by the presence of MSI in a tumor, and/or loss of expression in any of the MMR proteins. Twenty seven (22.9%) tumors were classified as MMR deficient, and 25 of these showed loss of protein expression (8 for MLH1/PMS2, 1 for isolated MLH1, 4 for isolated PMS2, 11 for MSH2/MSH6, and 1 for isolated MSH6). Clinicopathological features of patients with MMR deficiency are summarized in Table 2.

Nearly all cases of MSI had loss of protein expression; two cases with MSI retained normal expression of all four proteins. Likewise, 1 case with loss of MSH6 expression and one case with loss of PMS2 were MSS. The last patient was a 24 year-old woman who had CRC at age 15, a urothelial carcinoma at age 23, a metachronous CRC at age 24, and finally, a mediastinal B-cell lymphoma. Her CRC specimen showed loss of expression of PMS2 in tumor cells and in normal colonic surrounding tissue, leading to a presumptive diagnosis of constitutional MMR-deficiency syndrome due to bi-allelic PMS2 mutations.

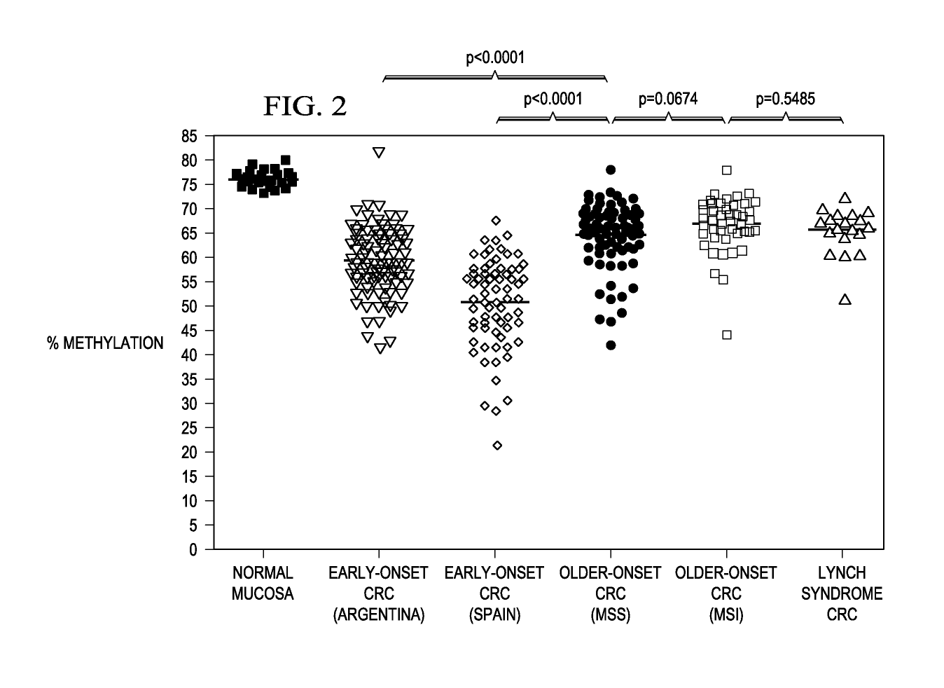

As shown in Table 1, compared to MMR-proficient tumors, MMR-deficient tumors were more likely to be located in the proximal colon (59.3% vs. 19.8%, p=0.0001), to be mucinous (63% vs. 26.4%, p=0.0001), to have tumor infiltrating lymphocytes (59.3% vs. 11.5%, p=0.0001), and to have MSI-suggestive pathology (81.5% vs. 47.2%, p=0.002). MMR deficient tumors were also more likely to be diagnosed at a lower stage (stages I-II: 51.9% vs. 29.7%, p=0.03), and to have less tumor recurrence or progression (22.2% vs. 44%, p=0.042). Although there was no difference in the age of CRC diagnosis between the 2 groups, the chance of having a MMR-deficient tumor was greater among younger patients (12-30 years: 8/27, 29.6%; 31-40 years: 9/45, 20%, 41-50 years: 10/46, 21.7%). Finally, patients with MMR-deficient tumors showed a trend towards a better 3 year overall survival (96.3% vs. 83.5%; p=0.1, log rank; FIG. 2).

Somatic BRAF mutation was present in one MMR-deficient tumor (Table 2). This case was a 49-year-old male with an MSI tumor in the cecum that showed loss of MLH1 and PMS2 protein expression. This case showed high degree of MLH1 promoter methylation (88%) and was therefore likely associated with CpG island methylator phenotype (CIMP)²⁴. In the rest of the MLH1-deficient tumors, presumably carriers of MLH1 germline mutations, 4 showed very low levels of methylation (range, 1-2%), and the other 4 showed intermediate levels (range, 25-51%).

TABLE 2
Clinicopathological and molecular features of patients with MMR deficiency

| | Age/ | | | Immunohistochemistry¹ | | | | | Other | Family | BRAF | MLH1 | LINE-1 |
|---|---|---|---|---|---|---|---|---|---|---|---|---|---|
| Case | Sex | Location | Stage | MSI | MLH1 | PMS2 | MSH2 | MSH6 | tumors | history² | status | methylation | methylation |
| 2ARG | 46 F | Ascending | II | MSI | | | | | colon | No | wt | | 53 |
| | | | | | | | | | (46) | | | | |
| 63ARG | 12 M | Rectum | IV | MSI | | | | | No | No | wt | | 53 |
| 90ARG | 19 M | Caecum | IV | MSI | | | | | No | No | wt | | 59 |
| 24ARG | 34 M | Hepatic | I | MSI | | | | | No | No | wt | | 69 |
| | | flexure | | | | | | | | | | | |
| 23ARG | 30 F | Sigmoid | III | MSI | | | | | No | No | ND | | 71 |
| 71ARG | 38 F | Rectum | II | MSI | | | | | No | Pancreas | wt | | 60 |
| | | | | | | | | | | (mother, 52) | | | |
| | | | | | | | | | | Colon | | | |
| | | | | | | | | | | (uncle, 55) | | | |
| | | | | | | | | | | Colon | | | |
| | | | | | | | | | | (cousin, 44) | | | |
| 16ARG | 52 F | Caecum | III | MSI | | | | | No | No | wt | | 63 |
| 97ARG | 54 F | Ascending | II | MSI | | | | | No | No | wt | | 59 |
| 84ARG | 56 M | Rectum | I | MSI | | | | | colon | Colon (sister, | wt | | 66 |
| | | | | | | | | | (40) | 37 and 49) | | | |
| | | | | | | | | | | Colon | | | |
| | | | | | | | | | | (father, 37) | | | |
| 62ARG | 57 F | Rectum | III | MSI | | | | | No | No | wt | | 49 |
| 76ARG | 53 M | Caecum | II | MSI | | | | | No | No | wt | | 51 |
| 77ARG | 59 F | Ascending | III | MSS | | | | | No | Colon | wt | | 62 |
| | | | | | | | | | | (grandmother, | | | |
| | | | | | | | | | | 65) | | | |
| 19ARG | 37 F | Caecum | II | MSI | | | | | No | No | wt | 2% | 66 |
| 101ARG | 43 F | Descending | II | MSI | | | | | No | No | wt | 27% | 64 |
| 46ARG | 42 M | Caecum | II | MSI | | | | | No | No | wt | 25% | 59 |
| 93ARG | 33 M | Splenic | II | MSI | | | | | No | No | wt | 1% | 61 |
| | | flexure | | | | | | | | | | | |
| 49ARG | 43 M | Caecum | II | MSI | | | | | No | No | wt | 26% | 63 |
| 37ARG | 49 F | Caecum | III | MSI | | | | | No | No | mutated | 88% | 64 |
| 38ARG | 50 M | Descending | III | MSI | | | | | No | No | wt | 2% | 66 |
| 115ARG | 55 F | Rectum | II | MSI | | | | | No | No | wt | 51% | 64 |
| 21ARG | 15 F | Caecum | III | MSS | | | | | No | No | wt | | 71 |
| 113ARG | 36 M | Descending | III | MSI | | | | | No | No | wt | | 47 |
| 18ARG | 58 M | Hepatic | II | MSI | | | | | No | No | wt | | 62 |
| | | flexure | | | | | | | | | | | |
| 79ARG | 51 M | Transverse | III | MSI | | | | | No | Colon | wt | | 61 |
| | | | | | | | | | | (aunt, 76) | | | |
| 108ARG | 41 M | Ascending | II | MSI | | | | | No | No | wt | 1% | 65 |
| 112ARG | 28 F | Rectum | III | MSI | | | | | No | No | wt | | 63 |
| 82ARG | 26 M | Caecum | III | MSI | | | | | No | Colon | wt | | 63 |
| | | | | | | | | | | (mother, 52) | | | |
| | | | | | | | | | | Colon | | | |
| | | | | | | | | | | (aunt, 46) | | | |

MMR: mismatch repair;
MSI: microsatellite instability;
MSS: microsatellite stability;
ND: not determined;
wt: wild-type
¹Solid cells indicate loss of protein expression,
²Affected relative and age at diagnosis are indicated between parentheses.

TABLE 5
Clinicopathological and molecular features of MUTYH mutation carriers.

| | | | Tumor | | | Mucinous | MSI | | Other | Synchronous | |
|---|---|---|---|---|---|---|---|---|---|---|---|
| Case | Age | Sex | location | Stage | Differentiation | production | status | MMR IHC | tumors | adenomas (number) | MUTYH mutation |
| 020ARG | 42 | Male | Splenic | IV | Moderately | No | MSS | Normal | No | No | G393D/— |
| | | | flexure | | | | | | | | |
| 064ARG | 39 | Male | Cecum | IIB | Moderately | Yes | MSS | Normal | Rectum | Yes (3) | G393D/— |
| | | | | | | | | | (41) | | |
| 074ARG | 29 | Female | Rectum | IIIA | Moderately | No | MSS | Normal | No | Yes (2) | Y176C/W472S |

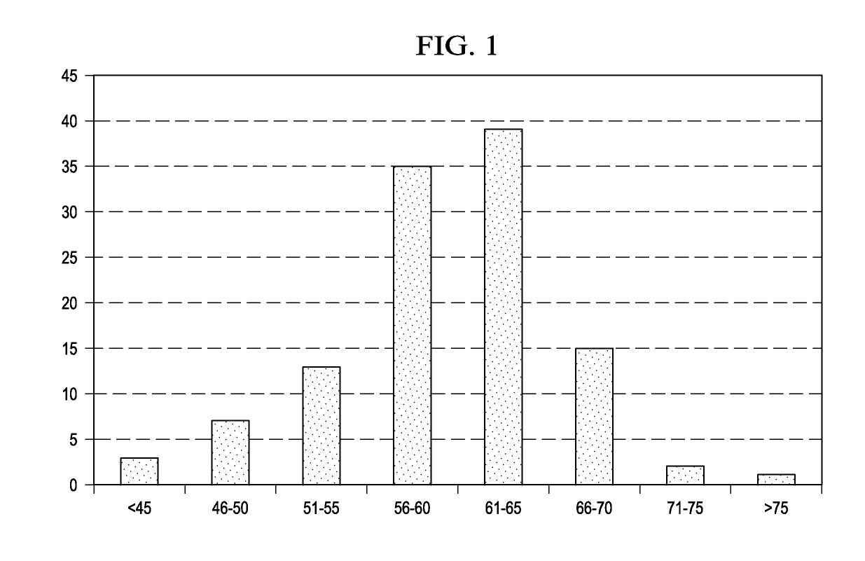

LINE-1 methylation analysis. A quantitative bisulfite pyrosequencing method was used to determine the methylation status of LINE-1 repetitive sequences in the CRCs. The average methylation in the CRCs was 59.97% (standard deviation, 6.57), which followed a normal distribution (FIG. 1). Clinicopathological features associated with LINE-1 methylation are shown in Table 3. A significant difference in LINE-1 methylation status was found according to tumor location, with lower levels of methylation in distal compared with proximal tumors (59.02% vs. 62.3%, p=0.015). In addition, a trend towards lower levels of methylation was found in females (58.87% vs. 60.93, p=0.092) and in non-mucinous tumors (59.24% vs. 61.41%, p=0.096). No differences in LINE-1 methylation status were found for any of the other clinicopathological features.

TABLE 3
LINE-1 methylation level in early-onset colorectal cancer

| Clinical, pathological or | Total | | Standard | |
|---|---|---|---|---|
| molecular features | (n) | Mean | deviation | p-value |
| Sex | | | | |
| Male | 61 | 60.93 | 6.598 | 0.092 |
| Female | 54 | 58.87 | 6.422 | |
| Age | | | | |
| ≧30 years | 91 | 60.26 | 6.757 | 0.345 |
| <30 years | 24 | 58.83 | 5.791 | |
| Body Mass Index (kg/m2) | | | | |
| <30 | 100 | 59.71 | 6.029 | 0.728 |
| ≧30 | 10 | 60.4 | 5.337 | |
| Family history of CRC¹ | | | | |
| Yes | 16 | 59.68 | 6.005 | 0.246 |
| No | 99 | 61.75 | 9.40 | |
| Tumor location | | | | |
| Proximal to splenic flexure | 33 | 62.3 | 7.126 | 0.015 |
| Distal to splenic flexure | 82 | 59.02 | 6.128 | |
| Synchronous or metachronous CRC | | | | |
| Yes | 5 | 64.2 | 6.76 | 0.141 |
| No | 110 | 59.77 | 6.52 | |
| TNM tumor stage | | | | |
| I-II | 40 | 59.63 | 6.054 | 0.687 |
| III-IV | 75 | 60.15 | 6.861 | |
| Tumor differentiation | | | | |
| Well or moderate | 97 | 60.03 | 6.555 | 0.926 |
| Poor | 15 | 60.2 | 6.527 | |
| Mucinous component | | | | |
| ≧50% | 39 | 61.41 | 5.959 | 0.096 |
| <50% | 75 | 59.24 | 6.824 | |
| Medullary growth pattern | | | | |
| Yes | 11 | 61.36 | 4.905 | 0.453 |
| No | 103 | 59.79 | 6.748 | |
| Crohn's reaction | | | | |
| Yes | 12 | 62.75 | 3.545 | 0.128 |
| No | 98 | 59.63 | 6.908 | |
| Tumor infiltrating lymphocytes | | | | |
| Yes | 26 | 60.88 | 5.443 | 0.447 |
| No | 85 | 59.74 | 7.004 | |
| Microsatellite instability | | | | |
| MSI | 25 | 59.72 | 6.717 | 0.454 |
| MSS | 90 | 60.84 | 6.053 | |
| Mismatch repair deficiency² | | | | |
| Yes | 27 | 61.26 | 6.137 | 0.244 |
| No | 88 | 59.7 | 6.680 | |

P value was calculated by t-test
¹Including first and second degree relatives
²MSI-H and/or loss of expression of MMR proteins by immunohistochemistry

The LINE-1 methylation levels in this series was compared with another independent cohort of patients with CRC diagnosed at <50 years of age recruited in Spain⁴, 2 groups of patients with sporadic CRC diagnosed >50 year-old categorized by the presence or absence of MSI (MSI, n=46; MSS, n=89), a group of Lynch Syndrome CRCs (n=20), and normal colonic mucosa from individuals without tumors (n=32) (FIG. 2 and Table 4). As expected, the average LINE-1 methylation levels in normal colonic mucosa were higher than in tumor tissues for all groups. LINE-1 methylation levels in early-onset CRCs was 59.9% (SD, 6.5) and 51.1% (SD, 9.2) for the Argentinian and the Spanish cohorts, respectively. The mean methylation level in the combined cohort of early-onset CRCs (n=185) was 56.6% (SD, 8.6). Interestingly, tumor LINE-1 methylation levels in the two independent cohorts of early-onset CRC were significantly lower than that observed in older-onset CRCs and Lynch syndrome tumors (Table 4), suggesting that this represents a unique feature of this subgroup of tumors (p<0.0001 for all comparisons). LINE-1 hypomethylation levels were similar in older-onset sporadic MSI tumors (67.1%, SD 5.5), Lynch syndrome CRCs (66.3%, SD 4.5), and sporadic MSS tumors (65.1%, SD 6.3).

TABLE 4
LINE-1 methylation results in different clinical subgroups

| | Mean % LINE-1 | | | |
|---|---|---|---|---|
| | methylation (SD) | Range | P-value¹ | P-value² |
| Normal colonic | 76.5 (1.5) | 73.5-80.2 | | |
| mucosa (n = 32) | | | | |
| Earlyonset CRC | 56.6 (8.6) | 22-82 | <0.0001 | |
| (n = 185) | | | | |
| Lynch syndrome | 66.3 (4.5) | 52.1-73.1 | <0.0001 | <0.0001 |
| CRC (n = 20) | | | | |
| Older onset | 67.1 (5.5) | 44.7-78.3 | <0.0001 | <0.0001 |
| sporadic MSI | | | | |
| (n = 46) | | | | |
| Older onset | 65.1 (6.3) | 42.5-78.4 | <0.0001 | <0.0001 |
| sporadic MSS | | | | |
| (n = 89) | | | | |

CRC, colorectal cancer; SD, standard deviation
Mann Whitney test was used to compare the LINE-1 values
¹Values for comparison between normal colonic mucosa and other groups of CRC.
²Values for comparison between early onset CRC (n = 185) and other groups of CRC.

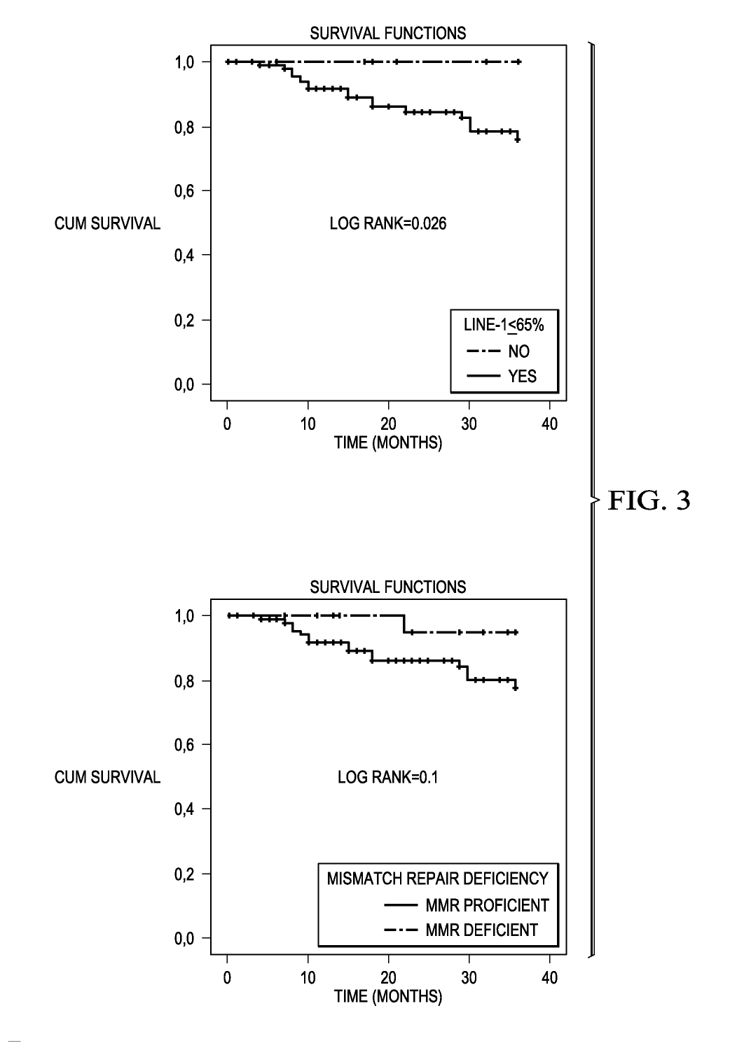

The effect of LINE-1 hypomethylation on the overall survival of CRC patients was evaluated. After evaluating different possible levels to distinguish these groups, it was found that in comparison to patients with ≦65% LINE-1 methylation, those with >65% LINE-1 methylation had significantly better overall survival (83.5% vs. 100%; p=0.026, log rank test; FIG. 3).

Germline MUTYH gene mutation analysis. Biallelic MUTYH mutations were found in 1/91 MMR-proficient cases (1.1%) (Table 5). This single case was a 29-year-old patient with a stage III rectal cancer and 2 synchronous adenomas. Two siblings of this patient had a history of attenuated polyposis and CRC (one presented with 30 adenomas and the other with 8 adenomas and an in situ carcinoma in the cecum); in both siblings total colectomies had been performed. Finally, two p.G393D heterozygous patients were identified that had no specific clinicopathological features.

Several studies have suggested that early-onset CRC constitutes a biologically distinct disease that is frequently associated with advanced stage, distal tumors, and poor prognosis^{2, 4, 5, 13}. The present inventors and others have shown that the known hereditary cancer syndromes only explain a minority of early-onset CRC cases; consequently, the pathogenic mechanism in the majority of cases remains unknown. This study aimed to gain further insight into the pathogenesis of early-onset CRC by assessing the clinicopathological and molecular features of 118 patients with early-onset CRC. The most interesting and novel result observed is that LINE-1 hypomethylation constitutes a unique feature of early-onset CRC patients, which was validated in two independent cohorts of patients. LINE-1 hypomethylation is a surrogate marker for genome-wide hypomethylation and is associated with increased chromosomal instability^{16, 17}; therefore, this may help some of the biological mechanisms underlying early-onset CRC. In addition, it was found that the frequency of MMR deficiency in this cohort is ˜20%, which is consistent with previous reports that characterized such populations⁴⁻⁶. Finally, it was found that MUTYH deficiency accounts for ˜1% of MMR-proficient CRCs.

Cancer is a complex disease, which arises as a result of both genetic and epigenetic alterations. Human CRCs often display changes in DNA methylation, and it has been known for decades that genome-wide hypomethylation is a consistent biochemical characteristic of human colorectal tumors^{16, 7, 25}. In mice, DNA hypomethylation is sufficient to induce T cell lymphomas²⁶. Genome-wide hypomethylation plays a causative role in cancer through different mechanisms: genomic instability, transcriptional activation of proto-oncogenes, activation of endogenous retroviruses and transposable elements, and the induction of inflammatory mediators. All these mechanisms have been associated with DNA hypomethylation, poor prognosis and tumor aggressiveness²⁶⁻³¹. Repetitive nucleotide elements, including long interspersed nucleotide elements-1 (i.e., LINE-1) contain numerous CpG sites, and prior studies have established that the level of LINE-1 methylation is an accurate indicator of cellular 5-methylcytosine content¹⁸, which reflects global DNA methylation. Consequently, LINE-1 methylation is frequently used as a surrogate for global DNA hypomethylation.

It has been suggested that LINE-1 methylation may identify different molecular subtypes of CRC. CIMP and MSI are inversely associated with DNA hypomethylation, suggesting that genomic hypomethylation represents an alternative pathway for CRC progression, and may reflect a fundamentally different disease process^{32, 33}. Moreover, LINE-1 hypomethylation has been associated with poorer survival among patients with CRC, and represents an independent factor for increased cancer-related mortality and overall mortality²⁰. Therefore, evaluation of tumoral LINE-1 methylation and its correlation with clinical and pathological features is important to determine the potential clinical value of this biomarker.

A quantitative pyrosequencing assay was used for LINE-1 methylation, which is a robust, accurate and reproducible method to precisely quantitate this in individual tumors¹⁸. Compared to older-onset colorectal tumors, the inventors found significantly lower levels of LINE-1 methylation in early-onset CRCs. This observation was validated in an independent set of early-onset CRC patients, reinforcing the strength of these conclusions. In addition, it was found that LINE-1 hypomethylation was associated with distal tumors and worse prognosis. Although there are no previous studies that have specifically examined LINE-1 methylation in early-onset CRC patients, a recent study suggested a relationship between greater LINE-1 hypomethylation in CRC and earlier onset of the cancer (<60 years)²¹. The present inventors recognized that the presence of a distinct subtype of CRC with a unique pathogenic mechanism^{4, 13}. Since the degree of LINE-1 hypomethylation is a prognostic marker in CRC and our data show that LINE-1 hypomethylation is a characteristic feature of early-onset CRC, this study provides a novel and previously unrecognized explanation for some of the biological differences involved in early-onset CRCs. In this regard, we are currently investigating whether LINE-1 hypomethylation causes direct transcriptional reactivation of certain proto-oncogenes in this setting, a unique feature that might help explain the aggressive clinical behavior of early-onset CRC.

Lynch syndrome is the most frequent hereditary cause of CRC, and accounts for approximately 1-3% of all CRCs'. It is an autosomal dominant condition caused by germline mutations in the DNA MMR genes (MLH1, MSH2, MSH6, PMS2), and MSH2 and MLH1 account for ˜90% of identifiable families. This syndrome has a gene-dependent variable penetrance for CRC and endometrial carcinoma, and an increased risk for various other extracolonic tumors. The diagnosis of Lynch syndrome has been traditionally based on tumor MMR deficiency analysis when this disease is suspected^{10, 11}, but the definitive diagnosis is established by finding a deleterious germline mutation in a DNA MMR gene. However, detecting Lynch syndrome is a particular challenge in the absence of a reliable family history. For this reason, universal screening with tumor MMR-deficiency analysis has been suggested^{34, 35}. The present inventors have previously shown that MMR deficiency accounts for up to 20% of early-onset CRC cases^{4, 5}, and also found that the pattern of MMR deficiency in early-onset CRC patients is not identical to that for all Lynch syndrome cases, and is characterized by in increased frequency of MSH6 and PMS2 deficiency. Another diagnostic challenge the MSH6-deficient CRC, as these might be missed if the screening algorithm relies entirely on MSI testing and does not include MMR immunohistochemistry²². In the present study, the MMR status in an Argentinian population of early-onset CRC was evaluated by analyzing both MSI and immunohistochemistry of the four MMR proteins. Twenty seven (22.9%) tumors were classified as MMR deficient. MSH2 and MLH1 deficiency accounted for the majority of cases, however, up to 20% were due to either MSH6 or PMS2 deficiency. One out of 9 MLH1-deficient cases had a BRAF mutation, which is typically associated with MLH1 promoter hypermethylation. In the rest of the MLH1-deficient cases, 4 had different degrees of MLH1 methylation, suggesting that promoter methylation might be the second hit in putative Lynch syndrome MLH1-type patients^{36, 37}. It is noteworthy that 2 patients had MSI tumors with normal DNA MMR protein expression, highlighting possible limitations when using either method, since these patients would not have otherwise been identified if immunohistochemistry had been used as the only screening technique. These results show that most patients with MMR-deficient tumors did not display any significant family history of CRC or other Lynch syndrome associated tumors. These facts underscore the importance of considering the diagnosis of Lynch syndrome in all early-onset CRC even in the absence of family history, given the important clinical implications for the management of affected individuals and their relatives³⁸.

Only found 1 case with biallelic germline mutations in the MUTYH gene was found (p.Y176C; p.W472S) in a 29-year-old female with no family history and a MSS rectal cancer. The p.W472S variant has not been previously described and is predicted to be probably damaging based on PolyPhen 2 software analysis³⁹. Therefore, novel and previously unrecognized MUTYH mutations should also be considered when evaluating early-onset CRC.

In summary, a large cohort of early-onset CRC cases was studied and it was found that LINE1 hypomethylation in these tumors constitutes a unique and specific feature compared with older-onset CRC, which is suggestive of a distinct molecular subtype of these colorectal neoplasms. These results show that the LINE-1 methylation status could be used as prognostic biomarker for young people with CRC. Future studies can be conducted to understand the mechanisms by which DNA hypomethylation affects CRC prognosis. In addition, it was found that MMR deficiency accounts for 1 in 5 cases of early-onset CRC. These results show that MMR-deficiency should be systematically evaluated in all cases with both MSI and abnormal DNA MMR immunohistochemistry, and that MUTYH germline mutations should be ruled out in MMR-proficient cases.

TABLE 5
Pyrosequencing primer description

| | | Reverse | | Product | Sequence |
|---|---|---|---|---|---|
| Assay | Forward | (5′biotinylated) | Sequencing | size | to analyze |
| MUTYH | ACACAGGAGG | CCAAGACTCCTG | GGAGGTGAATC | 118 | GGCTGGCCTGGGCTA/ |
| Y176C | TGAATCAACTC | GGTTCCTAC | AACTCTG | | GCTATTCTCGTGGCC |
| | TG | (SEQ ID NO: 2) | (SEQ ID NO: 3) | | GGCGGCTGCAG |
| | (SEQ ID NO: 1) | | | | (SEQ ID NO: 4) |
| MUTYH | GGCTGCCCTCC | AGGTCACGGAC | TGCCCTCCCTCT | 57 | G/ATCTGCTGGCAGGA |
| G393D | CTCTCA | GGGAACTC | CAG | | CTGTGGGAGTTC |
| | (SEQ ID NO: 5) | (SEQ ID NO: 6) | (SEQ ID NO: 7) | | (SEQ ID NO: 8) |
| BRAF | GAAGACCTCA | ATAGCCTCAATT | AGGTGATTTTGG | 122 | A/TGAAATCT |
| | CAGTAAAAAT | CTTACCATCC | TCTAGCTACAG | | (SEQ ID NO: 12) |
| | AG | (SEQ ID NO: 10) | (SEQ ID NO: 11) | | |
| | (SEQ ID NO: 9) | | | | |
| LINE-1 | TTTTGAGTTAG | AAAATCAAAAA | AGTTAGGTGTGG | 150 | TTYGTGGTGYGTYGT |
| | GTGTGGGATA | ATTCCCTTTC | GATATAGT | | TTTTTAAGTYGGTTTG |
| | TA | (SEQ ID NO: 14) | (SEQ ID NO: 15) | | AAAAGYGT |
| | (SEQ ID NO: 13) | | | | (SEQ ID NO: 16) |
| MLH1 | GAAATTTGATT | TCAACCAATCAC | TGATTGGTATTT | 119 | AATTAATAGTTGTYG |
| | GGTATTTAAGT | CTCAATACCTC | AAGTTGTTT | | TTGAAGGGTGGGGTT |
| | TGTTTAAT | (SEQ ID NO: 18) | (SEQ ID NO: 19) | | GGATGGYGTAAGTTA |
| | (SEQ ID NO: 17) | | | | TAGTTGAAGGAAGAA |
| | | | | | YGTGAGTAYGAGG |
| | | | | | (SEQ ID NO: 20) |

Y indicates C/T

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20110028332: Marker for Diagnosis of Breast Cancer, Test Method, and Test Kit.
U.S. Pat. No. 7,547,771: Polymorphic Markers of Prostate Carcinoma Tumor Antigen-1 (PCTA-1).
U.S. Patent Application No. 20090068660: Use of Methylated or Unmethylated Line-1 DNA As A Cancer Marker.
Jemal A, Siegel R, Ward E, et al. Cancer statistics, 2009. CA Cancer J Clin 2009; 59:225-49.
Siegel R L, Jemal A, Ward E M. Increase in incidence of colorectal cancer among young men and women in the United States. Cancer Epidemiol Biomarkers Prey 2009; 18:1695-8.
Dozois E J, Boardman L A, Suwanthanma W, et al. Young-onset colorectal cancer in patients with no known genetic predisposition: can we increase early recognition and improve outcome? Medicine (Baltimore) 2008; 87:259-63.
Giraldez M D, Balaguer F, Bujanda L, et al. MSH6 and MUTYH deficiency is a frequent event in early-onset colorectal cancer. Clin Cancer Res 2010; 16:5402-13.
Goel A, Nagasaka T, Spiegel J, et al. Low frequency of Lynch syndrome among young patients with non-familial colorectal cancer. Clin Gastroenterol Hepatol 2010; 8:966-71.
Gryfe R, Kim H, Hsieh E T, et al. Tumor microsatellite instability and clinical outcome in young patients with colorectal cancer. N Engl J Med 2000; 342:69-77.
Lynch H T, de la Chapelle A. Hereditary colorectal cancer. N Engl J Med 2003; 348:919-32.
Kastrinos F, Steyerberg E W, Mercado R, et al. The PREMM(1,2,6) model predicts risk of MLH1, MSH2, and MSH6 germline mutations based on cancer history. Gastroenterology 2011; 140:73-81.
Pinol V, Castells A, Andreu M, et al. Accuracy of revised Bethesda guidelines, microsatellite instability, and immunohistochemistry for the identification of patients with hereditary nonpolyposis colorectal cancer. Jama 2005; 293:1986-94.
Umar A, Boland C R, Terdiman J P, et al. Revised Bethesda Guidelines for hereditary nonpolyposis colorectal cancer (Lynch syndrome) and microsatellite instability. J Natl Cancer Inst 2004; 96:261-8.
Vasen H F, Watson P, Mecklin J P, et al. New clinical criteria for hereditary nonpolyposis colorectal cancer (HNPCC, Lynch syndrome) proposed by the International Collaborative group on HNPCC. Gastroenterology 1999; 116:1453-6.
Balaguer F, Castellvi-Bel S, Castells A, et al. Identification of MYH mutation carriers in colorectal cancer: a multicenter, case-control, population-based study. Clin Gastroenterol Hepatol 2007; 5:379-87.
Boardman L A, Johnson R A, Petersen G M, et al. Higher frequency of diploidy in young-onset microsatellite-stable colorectal cancer. Clin Cancer Res 2007; 13:2323-8.
Chan T L, Curtis L C, Leung S Y, et al. Early-onset colorectal cancer with stable microsatellite DNA and near-diploid chromosomes. Oncogene 2001; 20:4871-6.
Wolff E M, Byun H M, Han H F, et al. Hypomethylation of a LINE-1 promoter activates an alternate transcript of the MET oncogene in bladders with cancer. PLoS Genet; 6:e10009
Goelz S E, Vogelstein B, Hamilton S R, et al. Hypomethylation of DNA from benign and malignant human colon neoplasms. Science 1985; 228:187-90.
Jones P A, Baylin S B. The epigenomics of cancer. Cell 2007; 128:683-92.
Yang A S, Estecio M R, Doshi K, et al. A simple method for estimating global DNA methylation using bisulfite PCR of repetitive DNA elements. Nucleic Acids Res 2004; 32:e38.
Irahara N, Nosho K, Baba Y, et al. Precision of pyrosequencing assay to measure LINE-1 methylation in colon cancer, normal colonic mucosa, and peripheral blood cells. J Mol Diagn 2010; 12:177-83.
Ogino S, Nosho K, Kirkner G J, et al. A cohort study of tumoral LINE-1 hypomethylation and prognosis in colon cancer. J Natl Cancer Inst 2008; 100:1734-8.
Baba Y, Huttenhower C, NOsho K, et al. Epigenomic diversity of colorectal cancer indicated by LINE-1 methylation in a database of 869 tumors. Mol Cancer 2010; 9:125.
Goel A, Nagasaka T, Hamelin R, et al. An optimized pentaplex PCR for detecting DNA mismatch repair-deficient colorectal cancers. PLoS One 2010; 5:e9393.
Goel A, Xicola R M, Nguyen T P, et al. Aberrant DNA methylation in hereditary nonpolyposis colorectal cancer without mismatch repair deficiency. Gastroenterology 2010; 138:185462.
Weisenberger D J, Siegmund K D, Campan M, et al. CpG island methylator phenotype underlies sporadic microsatellite instability and is tightly associated with BRAF mutation in colorectal cancer. Nat Genet 2006; 38:787-93.
Feinberg A P, Gehrke C W, Kuo K C, et al. Reduced genomic 5-methylcytosine content in human colonic neoplasia. Cancer Res 1988; 48:1159-61.
Gaudet F, Hodgson J G, Eden A, et al. Induction of tumors in mice by genomic hypomethylation. Science 2003; 300:489-92.
Yamada Y, Jackson-Grusby L, Linhart H, et al. Opposing effects of DNA hypomethylation on intestinal and liver carcinogenesis. Proc Natl Acad Sci USA 2005; 102:13580-5.
Karpf A R, Matsui S. Genetic disruption of cytosine DNA methyltransferase enzymes induces chromosomal instability in human cancer cells. Cancer Res 2005; 65:8635-9.
Shahrzad S, Bertrand K, Minhas K, et al. Induction of DNA hypomethylation by tumor hypoxia. Epigenetics 2007; 2:119-25.
Rodriguez J, Frigola J, Vendrell E, et al. Chromosomal instability correlates with genome-wide DNA demethylation in human primary colorectal cancers. Cancer Res 2006; 66:84629468.
Esteller M. Epigenetics in cancer. N Engl J Med 2008; 358:1148-59.
Estecio M R, Gharibyan V, Shen L, et al. LINE-1 hypomethylation in cancer is highly variable and inversely correlated with microsatellite instability. PLoS One 2007; 2:e399.
Ogino S, Kawasaki T, Nosho K, et al. LINE-1 hypomethylation is inversely associated with microsatellite instability and CpG island methylator phenotype in colorectal cancer. Int J Cancer 2008; 122:2767-73.
Boland C R, Shike M. Report from the Jerusalem workshop on Lynch syndrome-hereditary nonpolyposis colorectal cancer. Gastroenterology 2010; 138:2197 e1-7.
Lindor N M, Petersen G M, Hadley D W, et al. Recommendations for the care of individuals with an inherited predisposition to Lynch syndrome: a systematic review. Jama 2006; 296:1507-17.
Ollikainen M, Hannelius U, Lindgren C M, et al. Mechanisms of inactivation of MLH1 in hereditary nonpolyposis colorectal carcinoma: a novel approach. Oncogene 2007; 26:45419.
Rahner N, Friedrichs N, Steinke V, et al. Coexisting somatic promoter hypermethylation and pathogenic MLH1 germline mutation in Lynch syndrome. J Pathol 2008; 214:10-6.
Jarvinen H J, Renkonen-Sinisalo L, Aktan-Collan K, et al. Ten years after mutation testing for Lynch syndrome: cancer incidence and outcome in mutation-positive and mutation-negative family members. J Clin Oncol 2009; 27:4793-7.
Adzhubei I A, Schmidt S, Peshkin L, et al. A method and server for predicting damaging missense mutations. Nat Methods 2010; 7:248-9.

What is claimed is:

1. A method for predicting, detecting, diagnosing or monitoring pre-cancer or cancer in a human subject comprising the steps of:
obtaining one or more biological samples from the human subject;
determining a LINE-1 methylation level for the one or more biological samples; and
comparing the LINE-1 methylation level to a LINE-1 methylation control level, wherein a lower degree of the LINE-1 methylation level is indicative of an early-onset colorectal cancer.

2. The method of claim 1, wherein the biological samples are selected from the group consisting of a tissue sample, a fecal sample, a cell homogenate, a blood sample, one or more biological fluids, or any combinations thereof.

3. The method of claim 1, wherein the LINE-1 methylation level is higher than an Alu methylation level.

4. The method of claim 1, wherein the LINE-1 methylation level is determined by amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing.

5. The method of claim 1, wherein the LINE-1 methylation level is determined by quantitative bisulfite pyrosequencing.

6. The method of claim 1, wherein the LINE-1 methylation level is determined by quantitative bisulfite pyrosequencing using the nucleic acids of SEQ ID NOS: 1 to 20.

7. A biomarker for predicting, detecting, diagnosing or monitoring an early-onset of colorectal cancer in a human subject comprising:
a biomarker to determine a methylation level of LINE-1, wherein a lower methylation level of LINE-1 is indicative of an early-onset colorectal cancer in the human subject.

8. A kit for determining an early-onset of colorectal cancer in a human subject comprising:
a biomarker detecting reagent for measuring a LINE-1 methylation level in a sample; and
instructions for the use of the biomarker detecting reagent in diagnosing the presence of early-onset of colorectal cancer, wherein the instructions comprise providing step-by-step directions to compare the LINE-1 methylation level in the sample with a LINE-1 methylation control level.

9. The kit of claim 7, wherein the sample is selected from the group consisting of a tissue sample, a fecal sample, a cell homogenate, a blood sample, one or more biological fluids, or any combinations thereof.

10. The kit of claim 7, wherein the LINE-1 methylation control level is obtained from the sample from a healthy subject, wherein the healthy subject is a human subject not suffering from early-onset colorectal cancer.

11. The kit of claim 7, wherein the LINE-1 methylation level is determined by amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing.

12. The kit of claim 7, wherein the detection is by quantitative bisulfite pyrosequencing.

13. The kit of claim 7, wherein the detection is by quantitative bisulfite pyrosequencing using the nucleic acids of SEQ ID NOS: 1 to 20.

14. A method for selecting a cancer therapy for a patient diagnosed with early-onset of colorectal cancer, the method comprising the steps of:
determining a methylation level of LINE-1 in a biological samples of the subject, wherein the methylation level of LINE-1 is indicative of early-onset of colorectal cancer; and
selecting the cancer therapy based on the determination of the presence of early-onset of colorectal cancer in the subject.

15. A method of performing a clinical trial to evaluate a candidate drug believed to be useful in treating early-onset of colorectal cancer, the method comprising:
a) determining the presence of an early-onset of colorectal cancer by a method comprising the steps of: determining an overall LINE-1 methylation level in one or more cells obtained from a biological sample of the subject, wherein a lower overall LINE-1 methylation level compared to a reference control is indicative of an early-onset of colorectal cancer;
b) administering a candidate drug to a first subset of the patients, and
a placebo to a second subset of the patients;
a comparable drug to a second subset of the patients; or
a drug combination of the candidate drug and another active agent to a second subset of patients;
c) repeating step a) after the administration of the candidate drug or the placebo, the comparable drug or the drug combination; and
d) monitoring a change in the overall LINE-1 methylation level as compared to any reduction occurring in the second subset of patients, wherein a statistically significant reduction indicates that the candidate drug is useful in treating said disease state.

16. A method for detecting a pre-cancer or an early-onset of colorectal cancer in a human subject comprising the steps of:
identifying the human subject suspected of suffering from a colorectal cancer;
obtaining one or more biological samples from the human subject;
determining a LINE-1 methylation level for the one or more biological samples; and
comparing the LINE-1 methylation level to a LINE-1 methylation control level, wherein a lower degree of the LINE-1 methylation level is indicative of an early-onset colorectal cancer.

17. The method of claim 16, wherein the biological samples are selected from the group consisting of a tissue sample, a fecal sample, a cell homogenate, a blood sample, one or more biological fluids, or any combinations thereof.

18. The method of claim 16, wherein the LINE-1 methylation level is higher than an Alu methylation level.

19. The method of claim 16, wherein the LINE-1 methylation level is determined by amplification of inter-methylated sites; bisulphite conversion followed by capture and sequencing; bisulphite methylation profiling; bisulphite sequencing; bisulphite padlock probes; high-throughput arrays for relative methylation; bisulphite restriction analysis; differential methylation hybridization; HpaII tiny fragment enrichment by ligation-mediated PCR; methylated CpG island amplification; methylated CpG island amplification with microarray hybridization; methylated DNA immunoprecipitation; methylated CpG immunoprecipitation; methylated CpG island recovery assay; microarray-based methylation assessment; methylation-sensitive arbitrarily primed PCR; methylation-sensitive cut counting; methylation-specific PCR; methylation-sensitive single nucleotide primer extension; next-generation sequencing; restriction landmark genome scanning; reduced representation bisulphite sequencing; or whole-genome shotgun bisulphite sequencing.

20. The method of claim 16, wherein the LINE-1 methylation level is determined by quantitative bisulfite pyrosequencing.

21. The method of claim 16, wherein the LINE-1 methylation level is determined by quantitative bisulfite pyrosequencing using the nucleic acids of SEQ ID NOS: 1 to 20.

22. A method for detecting pre-cancer or cancer in a human subject comprising the steps of:
obtaining one or more biological samples from the human subject;
processing the one or more biological samples to determine a LINE-1 methylation level for the one or more biological samples; and
comparing the LINE-1 methylation level from the one or more biological samples to a LINE-1 methylation level from a normal colorectal tissue, wherein a lower degree of the LINE-1 methylation level is indicative of an early-onset colorectal cancer.

23. A method of using a pharmacodynamic (PD) biomarker for determining a pharmacological response to a treatment of an early-onset of colorectal cancer, the method comprising:
determining an overall LINE-1 methylation level in one or more cells obtained from a first biological sample of a subject, wherein a lower overall LINE-1 methylation level compared to a normal sample from the subject that is not suspected of having cancer, is indicative of an early-onset of colorectal cancer;
administering a drug to the subject at a first time,
repeating the step of determining an overall LINE-1 methylation level in one or more cells obtained from a second biological sample from the subject at a second time; and
comparing the overall LINE-1 methylation at the first and the second time, wherein a statistically significant reduction in LINE-1 methylation indicates that the drug is useful in treating the early-onset of colorectal cancer.


SEQUENCE LISTING (20 sequences)

SEQ ID NO: 1 - DNA, 23 bp, artificial sequence
  synthetic oligonucleotide
acacaggaggtgaatcaactctg

SEQ ID NO: 2 - DNA, 21 bp, artificial sequence
  synthetic oligonucleotide
ccaagactcctgggttcctac

SEQ ID NO: 3 - DNA, 18 bp, artificial sequence
  synthetic oligonucleotide
ggaggtgaatcaactctg

SEQ ID NO: 4 - DNA, 40 bp, artificial sequence
  synthetic oligonucleotide
ggctggcctgggctrctattctcgtggccggcggctgcag

SEQ ID NO: 5 - DNA, 17 bp, artificial sequence
  synthetic oligonucleotide
ggctgccctccctctca

SEQ ID NO: 6 - DNA, 19 bp, artificial sequence
  synthetic oligonucleotide
aggtcacggacgggaactc

SEQ ID NO: 7 - DNA, 15 bp, artificial sequence
  synthetic oligonucleotide
tgccctccctctcag

SEQ ID NO: 8 - DNA, 26 bp, artificial sequence
  synthetic oligonucleotide
rtctgctggcaggactgtgggagttc

SEQ ID NO: 9 - DNA, 22 bp, artificial sequence
  synthetic oligonucleotide
gaagacctcacagtaaaaatag

SEQ ID NO: 10 - DNA, 22 bp, artificial sequence
  synthetic oligonucleotide
atagcctcaattcttaccatcc

SEQ ID NO: 11 - DNA, 23 bp, artificial sequence
  synthetic oligonucleotide
aggtgattttggtctagctacag

SEQ ID NO: 12 - DNA, 8 bp, artificial sequence
  synthetic oligonucleotide
wgaaatct

SEQ ID NO: 13 - DNA, 23 bp, artificial sequence
  synthetic oligonucleotide
ttttgagttaggtgtgggatata

SEQ ID NO: 14 - DNA, 21 bp, artificial sequence
  synthetic oligonucleotide
aaaatcaaaaaattccctttc

SEQ ID NO: 15 - DNA, 20 bp, artificial sequence
  synthetic oligonucleotide
agttaggtgtgggatatagt

SEQ ID NO: 16 - DNA, 39 bp, artificial sequence
  synthetic oligonucleotide
ttygtggtgygtygttttttaagtyggtttgaaaagygt

SEQ ID NO: 17 - DNA, 30 bp, artificial sequence
  synthetic oligonucleotide
gaaatttgattggtatttaagttgtttaat

SEQ ID NO: 18 - DNA, 23 bp, artificial sequence
  synthetic oligonucleotide
tcaaccaatcacctcaatacctc

SEQ ID NO: 19 - DNA, 21 bp, artificial sequence
  synthetic oligonucleotide
tgattggtatttaagttgttt

SEQ ID NO: 20 - DNA, 73 bp, artificial sequence
  synthetic oligonucleotide
aattaatagttgtygttgaagggtggggttggatggygtaagttatagttgaaggaagaaygtgagtaygagg